(19) United States
(12) Patent Application Publication
(10) Pub. No.: US 2008/0195056 A1
(43) Pub. Date: Aug. 14, 2008

(54) Apparatus And Method For Injecting A Pharmaceutical

(76) Inventors: Steven Bishop, Mountain View, CA (US); Adrian Benton James, Palo Alto, CA (US); John W. Lai, San Bruno, CA (US); Robert Ian Lister, Sunnyvale, CA (US); Nancy J. Serpiello, Emerald Hills, CA (US)

Correspondence Address: ELI LILLY & COMPANY, PATENT DIVISION, P.O. BOX 6288, INDIANAPOLIS, IN, 46206-6288, US

(21) Appl. No.: 11/916,929

(22) PCT Filed: Jun. 19, 2006
(86) PCT No.: PCT/US2006/023929

§ 371 Date: Dec. 7, 2007

Related U.S. Application Data
(60) Provisional application No. 60/692,437, filed on Jun. 21, 2005.
(60) Provisional application No. 60/695,048, filed on Jun. 29, 2005.

(51) Int. Cl. A61M 5/315

(52) U.S. Cl. 604/218

(57) ABSTRACT

A method and apparatus for administering a pharmaceutical. The method employs a delivery device including a housing, a pharmaceutical containing needled syringe movable within the housing, an activation button disposed at one of the housing, and wherein the housing is flared radially outward at the other end and designed to allow visibility of the needled syringe. A skin-contacting surface of the housing at the flared end is designed to limit slippage along the skin, and at least one injection targeting guide is provided. When the device is sited for injection, and without pressing the delivery device housing toward the injection site with any predetermined force by the one hand holding the housing, the activation button may be plunged with the other hand toward the housing to trigger an advancing assembly within the device that first automatically advances the needled syringe to insert a needle into the injection site, and that second automatically advances the syringe piston to force pharmaceutical through the inserted needle.

### BACKGROUND OF THE INVENTION

The present invention pertains to pharmaceutical delivery devices, and, in particular, to a delivery device that automatically injects a pharmaceutical.

Patients suffering from a number of different diseases frequently must inject themselves with pharmaceuticals. As some patients find it difficult to insert a needle of an injector into one's skin, and/or to operate the injector to inject the pharmaceutical through an inserted needle, a variety of devices have been proposed to facilitate these tasks.

One type of device automatically inserts a needle and then automatically injects a dose of medication through that inserted needle. With one known version of this type device, after unlocking the device by manually twisting a handle, a patient needs to position the device against an injection site, and then operate a trigger of the device by pressing the device firmly against the site. By such firm pressing, the hand graspable housing shifts toward the injection site, allowing the portion of the device placed and remaining against the skin to slide, relative to the shifting housing from which it proximally extends, distally into the housing to trigger the automatic needle insertion into the skin, and automatic injection through that inserted needle into the patient.

Another version of this type device requires a similar user interaction other than the twist unlocking, and further requires an additional safety button pressing during the firm pressing of the device to trigger the insertion and injection.

While these devices, as well as other proposed devices, may be useful for some people, other users may find different aspects of the design of these devices to be troublesome. For example, where activation requires pressing the entire device toward the injection site, whereby the hand grasping the housing must move relative to that injection site, some users may find such controlled hand movement disconcerting or difficult. Still further, users may worry with some proposed devices about properly stabilizing the device, in that the device may slip along the skin or the needle may not remain properly oriented during the entire injection process. Still further, some users may find it difficult with some proposed devices to inject accurately into precise locations that they have pre-selected.

Thus, it would be desirable to provide a device that can overcome one or more of these and other shortcomings of the prior art.

### BRIEF SUMMARY OF THE INVENTION

In one form thereof, the present invention provides a method of administering a pharmaceutical, including the step of: providing a delivery device including a housing having a first end and a second end spaced in an axial direction, a pharmaceutical containing needled syringe mounted within the housing to be movable in the axial direction from a first position to a second position, wherein an injection tip of the syringe needle is recessed within the housing when in the first position and projects from the housing beyond the first end when in the second position, the syringe having a piston advanceable toward the first end to force pharmaceutical contained in the syringe through the needle injection tip, wherein a region of the axial length of the housing proximate the first end is flared radially outward toward the first end, and is at least one of transparent and apertured to allow visibility of the needled syringe, wherein a skin-contacting surface of the housing first end includes a first region and a second region, the first region comprising a material having a greater coefficient of friction than a material of the second region to limit slippage along skin, and at least one injection targeting guide at the housing first end. The method further includes the steps of holding a portion of the housing with one hand and placing the delivery device with the skin-contacting surface of the housing against the skin such that the least one targeting guide aligns with an intended injection site, operating an unlocking system by rotating a part of the delivery device relative to the held portion of the housing, whereby an activation button disposed at the second end shifts axially away from the housing, and, without pressing the delivery device housing toward the injection site with any predetermined force by the one hand holding the housing portion, with another hand plunging the activation button toward the housing in the axial direction to trigger an advancing assembly within the device that first automatically advances the needled syringe from the first position to the second position, and that second automatically advances the syringe piston toward the second end.

One advantage of the present invention is that a medication delivery device may be provided which is intuitive and easy to operate.

Another advantage of the present invention is that a medication delivery device may be provided which does not require the entire apparatus be shifted after being properly sited for an intended injection.

Yet another advantage of the present invention is that a medication delivery device may be provided with a grip feature for engaging the skin around an injection site to limit slippage of the device during siting and injection.

Yet another advantage of the present invention is that a medication delivery device may be provided with targeting guides to assist a user in confirming the injection is to be delivered where desired.

Still another advantage of the present invention is that a medication delivery device may be provided which has an enlarged end to stabilize the device over an injection site, which enlarged end is transparent or otherwise configured to allow visibility of components such as a needle disposed therein, and which enlarged end may facilitate application of axial force against the injection site.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a front perspective view of a pharmaceutical delivery device of the present invention, wherein a cap of the device is shown mounted to the proximal end of the device housing;

FIG. 2 is a perspective view of the pharmaceutical delivery device of FIG. 1 with the cap removed, and after the device has manipulated to an unlocked condition;

FIG. 3 is a diagrammatic view in longitudinal cross-section of one suitable configuration of internal components of a delivery device of the present invention;

FIG. 4 is a diagrammatic view in longitudinal cross-section of another suitable configuration of internal components of a delivery device of the present invention in a ready state;

FIG. 5 is a diagrammatic view of the device of FIG. 4 at a point after activation at which the needle has been automatically inserted but before automatic injection has occurred;

FIG. 6 is a diagrammatic view of the device of FIG. 5 after automatic injection has occurred; and

FIG. 7 is a schematic showing additional details of a flared housing end.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

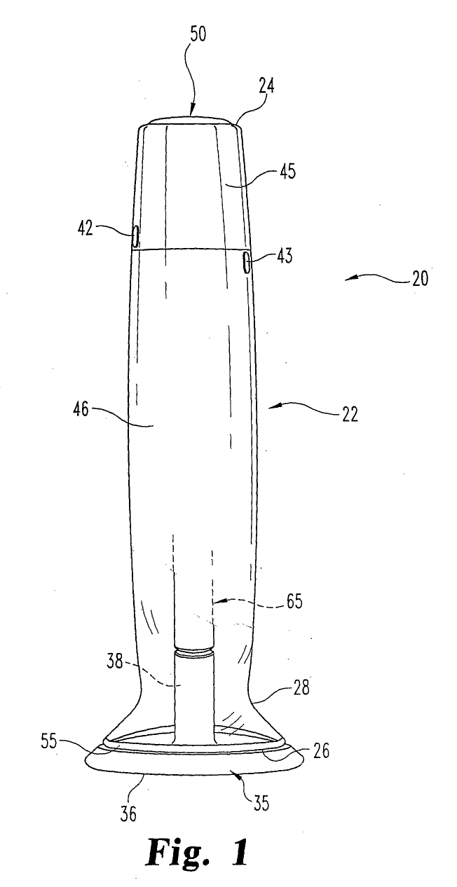

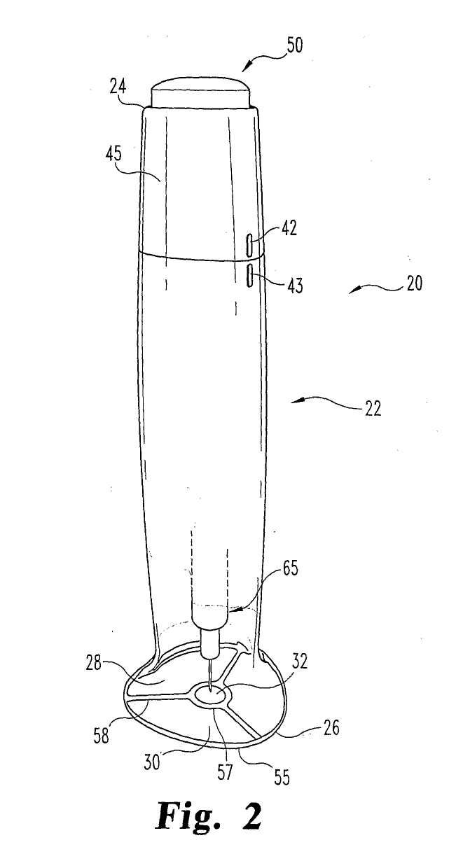

Referring now to FIGS. 1 and 2, there is shown a first embodiment of a pharmaceutical delivery device of the present invention. Any directional references in this detailed description with respect to the Figures, such as up or down, or top or bottom, are intended for convenience of description, and by itself does not limit the present invention or any of its components to any particular positional or spatial orientation.

The delivery device, generally designated 20, is designed to allow a user, with one hand on the device, to comfortably position the device on the skin at a pre-selected site before initiating the injection sequence. After such siting, the user's hand on the device can remain motionless, while keeping the device stable and comfortable, as needle insertion and injection can be activated with the other hand. The hand holding the device need not firmly press the entire device toward the injection site with any required trigger unlocking or trigger firing pressure. Device 20 provides feedback that needle insertion will take place at the intended location. The interaction strategy of the user with device 20 aids in providing accurate targeting of insertion, as well as stability and comfort during the injection procedure. Device 20 is single use delivery device based on a standard prefilled syringe primary containment. The injector system is delivered ready to use and provides a single fixed dose delivery.

The delivery device, generally designated 20, includes an outer housing 22 having a distal end 24 and a proximal end 26. As used herein, distal and proximal refer to axial locations on the delivery device relative to an injection site when the device is oriented for use at such site, whereby, for example, proximal end of the housing refers to the housing end that is closest to such injection site.

The exterior periphery of outer housing 22 is sized, shaped and/or constructed of materials to facilitate being gripped within one hand by a user or a caregiver during site selection and injection, and may accommodate reduced dexterity in users such as rheumatoid arthritis patients. The shown housing periphery is described herein as having a plastic construction that changes in size but not overall shape along its axial length. The largest transverse dimension or girth of housing 22, other that at its flared base, is between 20 and 30 millimeters, such as about 25 millimeters. Along its entire axial length, the shown housing exterior periphery is non-circular and lobular in transverse cross-sectional shape. The tri-lobular shape shown, which may be held by and within the space between digits of a user's hand, may be replaced with other shapes known in the art, including circular or rectilinear shapes, or shapes having fewer or additional lobes, within the scope of the invention. Other configurations, such as a less uniform shaping along its length, or a construction including a soft touch or rubber-like gripping layer covering a more rigid base, may be employed within the scope of the invention.

The proximal region or base 28 of the exterior periphery is flared outward relative to the housing to provide for stabilization and to enhance the user's application of a small downward force on the device housing during siting and injection. This downward force promotes stability against tipping and promotes locating the device by increasing friction forces that limit slippage along the skin. The shown flared region 28 starts at an axial position of about fourteen millimeters from proximal end 26, and ends at an axial position that is adjacent proximal end 26. Adjacent, as user herein with respect to the shown flare, means that the flaring ends no more than one millimeter from the injection site skin-contacting surface 30. Due to the housing being tapered slightly inward leading into the flared region 28 for the shown embodiment, the flared region 28 starts at the inflexion point of the axial contour. In alternate embodiments, the shown flared region 28 can begin flaring at a point which is at the proximal end of a non-tapering or uniformly shaped axial segment of the housing, or a slightly tapering outward housing body where the flared end is more pronounced. Along its axial length, flared region 28 is shown bowed in or concave.

The shown shaping and axial positioning of flared region 28 provides for a more ready and comfortable holding of the housing that allows a user to provide an axial force in the proximal direction without requiring large gripping forces being applied by the user. The flaring is of such as size to provide a proximal end face having a larger area that allows device 20 to be stabilized more easily by a user at the injection site to facilitate injection confidence. Although the maximum transverse dimension of the shown flared end is larger than the maximum transverse dimension of the rest of the housing, a flared end smaller than the maximum transverse dimension of some other part of the housing may be employed within the scope of the invention.

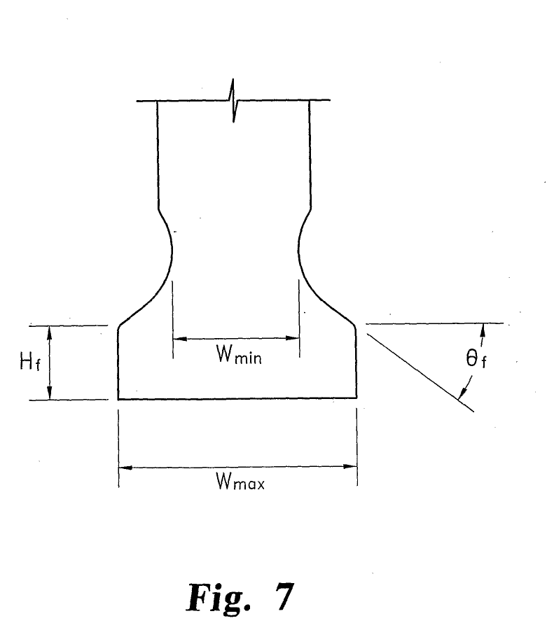

With additional reference to FIG. 7, there is schematically shown a type of flared housing end closely related to the concave housing shown with respect to FIGS. 1 and 2, and which is provided with several dimensions.

The height from the proximal end of the flare (H_{f}), relative to the proximal end of the housing, is from 0-20 mm, more preferably from 0-10 mm, and most preferably 0-5 mm. The difference in the transverse width of the largest dimension of the flared end, and the transverse width of the smallest dimension of the flared end, (Wₘₐₓ Wₘᵢₙ) is within the range of 5-50 mm, and more preferably the range of 10-30 mm, and preferably around 15 mm. The angle the proximal end of the flaring makes with horizontal (Of) is within the range of 0-70 degrees. Naturally, this angle refers to the flare prior to any convex radiusing at the outer periphery that softens its edge.

Other flared end shapings may be provided within the scope of the invention to facilitate handling of the device during siting and injecting. These flaring include circumferential flanges at or near the proximal end of the housing, or flarings that are convex instead of concave.

The injection site skin-contacting surface 30 of the housing is formed of a circumferential flange that inwardly projects from the flared region 28 at housing proximal end 26. The flange 30 defines a central aperture 32, aligned with the longitudinal axis of device 20, through which a needle is moved from within housing 22 during insertion.

As best shown in FIG. 2, skin-contacting surface 30 is provided with an over-molded ring 55 around the entire perimeter of the surface. Ring 55 is formed of a higher friction material, such as a Shore A, 50 durometer thermoplastic elastomer, which serves to engage the user's skin against which it is placed around the injection site to reduce the chance of device 20 moving with respect to the patient's body during the insertion and injection process. Readily visible guides, such as of a change in color or transparency, and such as a ring 57 around aperture 32, and radially extending lines 58 aligned with the housing lobes, provide a targeting feature that allow a user to accurately see where the needle is to enter the skin. In the preferred embodiment, targeting guides 57 and 58 are overmolded to surface 30 of the same gripping material as is ring 55 to further limit slippage. The proximal end region of housing 22 is preferably transparent around its entire circumference to allow visibility of the targeting guides 57, 58 to facilitate the user precisely locating the intended needle insertion, as well as visibility as necessary for a user to confirm a full syringe of medication prior to using device 20, and to confirm that all the medication has been delivered and that the needle has safely retracted into the housing after injection.

A needle cap 35 that covers aperture 32 is diagrammatically shown in FIG. 1 and is designed to be removed with lower dexterity or grip. Cap 35 includes a base 36 and an upstanding, blind bored member 38. Base 36 covers all of flange 30 and is sized to laterally project beyond the perimeter of the housing proximal end 26. Sealing member 38 fits through aperture 32 and sealing fits around the syringe needle to limit contamination. Rather than the shown one-piece cap construction, member 38 may be an existing rubber needle cap that comes as part of the primary container closure system, while base 36 may be a plastic part that fits around and engages member 38. Cap 35 can be removed from the covering arrangement shown in FIG. 1 by pulling it off the housing completely manually, or, for example, by pulling device 20 away as the cap 35 is braced against a flat surface, such as a table. The base 36 may be overmolded with an elastomeric material to make easier the gripping of the cap.

At the distal end of device 20, an activation button 50 for the automatic needle insertion and pharmaceutical injection is provided. In the shown embodiment, the top of activation button 50 is initially generally flush with the top of the device housing, and the device is locked. Such locking is indicated to the user by the vertical non-alignment of markings 42 and 43 on the lock sleeve 45 of the housing and the housing main body 46, respectively, but other locking indicating systems, such as plastic features, may be employed. When lock sleeve 45 sleeve is rotated such that markings 42 and 43 vertically align, button 50 pops up distally, and device 20 is unlocked and ready to use as shown in FIG. 2. Lock sleeve 45 is designed to be large and formed of a grippable material, such as a rubber coated plastic sleeve, to be twistable by users with limited dexterity. A coloring of the radial periphery along the height of button 50 makes noticing that the button is popped out easier for the user. The safety lock system is located at the distal end of device 20 to facilitate easy activation while the device is being held on the body with the other hand. The activation button is designed to facilitate use by patients with sensitive hands and limited dexterity. The shape and size of the button allow gross activation motion by the palm of a hand rather than the tips of fingers.

In alternate embodiments, the activation button can be a piece that is directly rotated to unlock, and plungable proximally to activate. One such variation may involve an activation button that has an outer periphery coextensive with the housing that it faces, which button, when rotated to unlock, shifts distally, and from that distal position can be plunged proximally to effect activation.

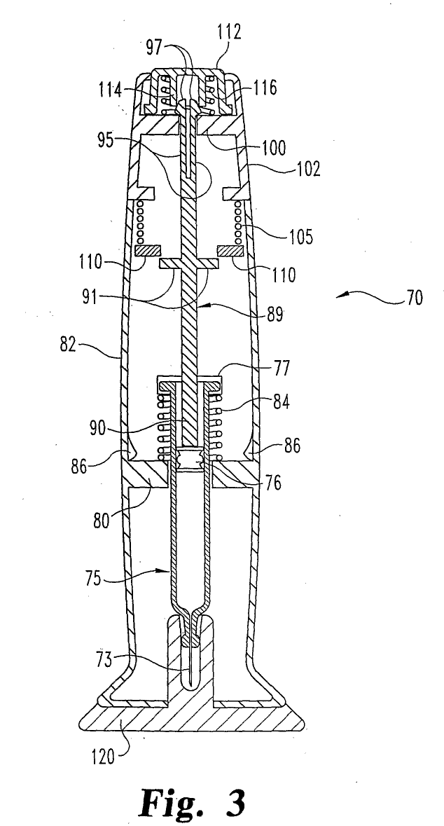

Various manners of providing a pop up button may be employed within the scope of the invention. The button may be designed to pop up at the moment when the lock sleeve rotation to the unlocked arrangement is essentially completed, or when such lock sleeve rotation is commenced, or at a time therebetween. In the case where the button is to pop, up only at the end of the unlocking rotation of the lock sleeve, such design may be accomplished by the button being configured to rotate with the lock sleeve during such motion, and at the end of such lock sleeve rotation, the button being biased to further rotate and cam upward along the lock sleeve to an operational position at which its subsequent plunging produces proper operation of the internal workings of the device. If the button is to pop out near the commencement of lock sleeve rotation, such button can be configured, for example, as described further below with respect to the embodiment of FIG. 3. In addition, in alternate embodiments, the button need not pop up or move when unlocked.

When activation button 50 is plunged, a needled syringe, generally designated 65, within housing is automatically driven proximally, causing the needle of that syringe to pass through flange aperture 32 and insert into the injection site. Delivery of the contents of the syringe then automatically proceed as a piston of the syringe is driven proximally within the syringe to force the contents through the inserted needle.

After needle insert and injection is complete, device 20 is configured to automatically withdraw the needle inside the device housing 22, indicating to the user the completion of the injection as well as shielding the needle from the user.

The components of delivery device 20 which are internal to housing 22 may be provided in a variety of manners to achieve the functionality described above. A wide assortment of automatic injecting mechanisms to first automatically insert a needle and to then automatically inject medicine through that needle are known and may be selectively employed. For example, a compressed spring or system of springs, possibly including a damping system, such as an annular foam member, to reduce noise and vibration at the end of insertion travel, may be employed. Similarly, a wide assortment of mechanisms to retract a needled syringe within the housing, or to extend a needle shield over the extended needled syringe, after the pharmaceutical within that syringe has been injected are also known and may be employed.

Referring now to FIG. 3, there is shown diagrammatically the internal workings of one delivery device within the scope of the present invention. The delivery device 70 includes a needled syringe 75 of a standard type mounted to a carriage 77. Syringe 75 is axially slidable within a guide collar 80 within the housing body 82. Carriage 77 is spring-biased upwardly by a helical spring 84. A pair of retraction ramps 86 are formed on the interior of the housing body 82 distally of guide collar 80. A plunger 89 that is rotatably fixed with respect to the housing extends longitudinally within the device and has a proximal end 90 that abuts the piston 76 of the syringe 75. The distal end of plunger 89 includes a pair of resilient fingers 95 with latch ends 97 that fit through and secure to an apertured flange 100 of a safety lock sleeve 102 that is rotatably mounted to the housing body 82. An insertion and delivery spring 105 has one end that abuts the housing, such as an inturned lip of sleeve 102, and an opposite end that abuts collar 110. Collar 110 includes a not shown portion that rides within a not shown track in the housing body. Collar 110 has a proximal face that, at a first angular orientation of the collar relative to the housing body, abuts radially projecting ears 91 of the plunger 89.

An activation button 112 is mounted within a hollow of the distal portion of sleeve 102. Button 112 includes a depending flange 114 having a portion not shown in FIG. 3 which engages the latches 97 when button 112 is rotated into a plungable position described below. Such not shown portion of flange 114 may be in the same radial position and depend farther proximally than the flange portion shown, or may be of the same height but radially closer to the axis of the device than the flange portion shown. The shown portion of flange 114 does not itself engage the latches, but rather helps to locate spring 116 and provide rigidity to the button.

To inject, and after cap 120 has been manually removed, when safety lock sleeve 102 is rotated relative to the housing body by a user, activation button co-rotates therewith and is released from a restraining engagement with a not shown finger of the housing, which release allows spring 116 to force activation button 112 distally. When sleeve 102 has been fully rotated, the farther depending or radially inward not shown portion of flange 114 is oriented such that when button 112 is subsequently manually plunged, latch ends 94 are abutted and squeezed inward to allow passage through flange 102. Spring 105 then drives collar 110, and therefore plunger 89, proximally, which first causes syringe needle 73 to project proximally from the housing and insert into a user, and then forces medication from the cartridge through the inserted needle. After plunger 89 has been shifted sufficiently proximally to cause an appropriate injection, collar 110 reaches the axial position at which camming ramps 86 are located, which ramps rotate collar 110 into an angular orientation that allows ears 91 to pass through collar. At this time, spring 84, which is weaker than spring 105, is free to force carriage 77 and the needle syringe 75 and plunger 89 distally through the collar 110 until the needle tip is withdrawn into housing body 82.

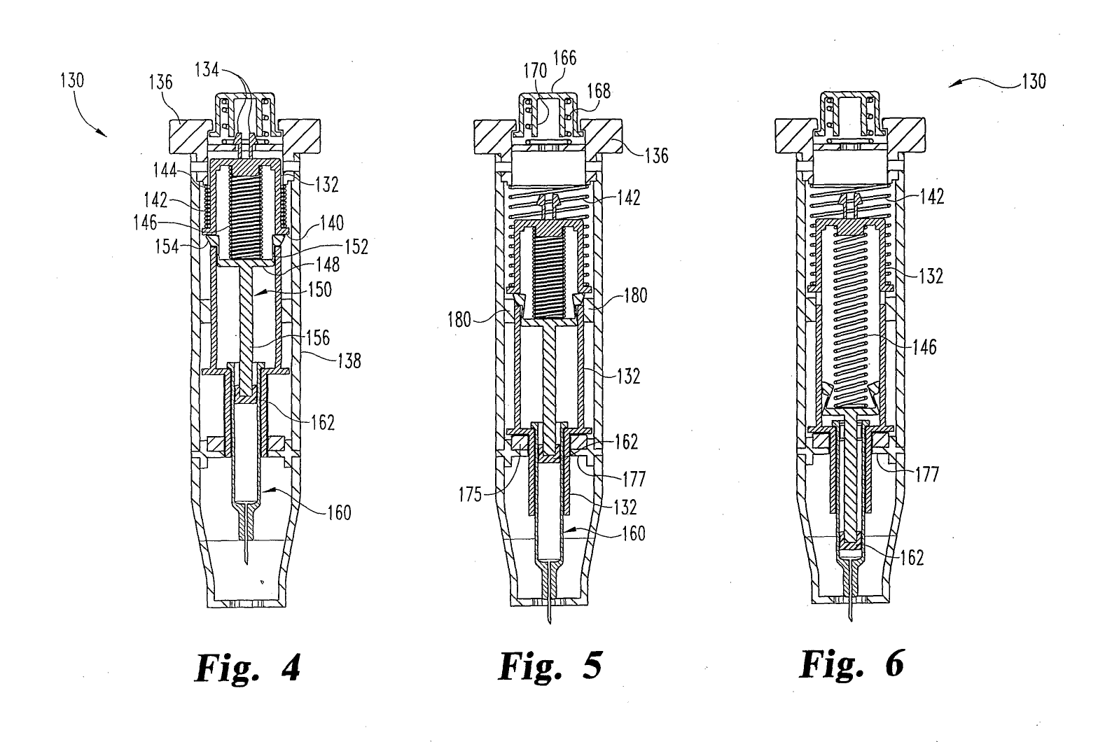

In another embodiment shown in various stages of its operation in FIGS. 4-6, a device 130 of the present invention is shown which, when triggered by a single plunging of an activation button, utilizes one spring to cause a needle insertion and a second spring to cause an injection through that needle. Device 130 is not shown with the trigger button that springs up when unlocked, or a flared proximal end, or as having a needle retraction after injection is complete, but one or more of such may be provided within the scope of the invention if desired.

In this embodiment, carriage 132 is releaseably retained by flexible latches 134 that extend through an opening in a housing cap 136 that is fixedly secured to housing body 138. Carriage 132 includes a shoulder 140 that radially projects outward. Insertion spring 142 is compressed between the housing at 144 and shoulder 140. Within a hollow interior of carriage 132, an injection spring 146 is disposed between an upper end of the carriage and a disk portion 148 of a plunger 150. A pair of plunger arms 152 with cammable latches 154 at their ends extend distally and radially from disk portion 148. Latches 154 extend through openings in the side wall of carriage 132. By the engagement of latches 154 with the carriage wall, plunger 150 is prevented from shifting proximally relative to the carriage. Plunger 150 includes a rod 156 that depends from disk portion 148 and axially extends and inserts within a needled syringe 160 to abut syringe piston 162. The barrel and needle of syringe 160 is mounted in the proximal end of carriage 132 to be movable therewith. When the delivery device 130 is in a ready condition, it is arranged as shown in FIG. 4.

When a user wishes to utilize delivery device 130, a pressing of the trigger button 166 that is retained in housing cap 136 causes button flange 170 to cam inwardly the latches 134. Spring 168 biases button 166 distally to keep flange 170 clear of latches 134 until a button plunging occurs. This inward movement of the latches releases carriage 132 to allow insertion spring 142 to drive the carriage proximally. During this driving motion, the needled syringe 160 moves as a unit with the carriage, but the medicine is not ejected from the syringe as plunger 150 remains axially fixed relative to the carriage due to its latching engagement therewith. Spring 142 continues to drive proximal motion until the housing with a damping member 175, such as a ring-shaped foam or elastic element disposed on housing shoulder 177, halts the carriage motion with the needle of syringe 160 extending through the opening of the housing at its proximal end.

Immediately before the carriage is so halted, however, plunger latches 154 reach a point at which displacement triggers 180 mounted to or formed within the housing body are abutted by the latches. This abutment forces latches 154 radially inward so as to be forced inward into the interior of the carriage when the injection spring 146 begins to drive plunger 150 proximally. At this point, device 130 is arranged as shown in FIG. 5.

Spring 146 continues to drive plunger 150 proximally and thereby drive the syringe piston 162 proximally to force medication from the syringe. When spring 146 has completed its extension such that the stroke of the plunger is completed, delivery device 130 is arranged as shown in FIG. 6.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

We claim:

1. A method of administering a pharmaceutical, comprising the steps of:
providing a delivery device including;
a housing having a first end and a second end spaced in an axial direction;
a pharmaceutical containing needled syringe mounted within the housing to be movable in the axial direction from a first position to a second position, wherein an injection tip of the syringe needle is recessed within the housing when in the first position and projects from the housing beyond the first end when in the second position, the syringe having a piston advanceable toward the first end to force pharmaceutical contained in the syringe through the needle injection tip;
wherein a region of the axial length of the housing proximate the first end is flared radially outward toward the first end, and is at least one of transparent and apertured to allow visibility of the needled syringe;
wherein a skin-contacting surface of the housing first end includes a first region and a second region, the first region comprising a material having a greater coefficient of friction than a material of the second region to limit slippage along skin; and
at least one injection targeting guide at the housing first end;
holding a portion of the housing with one hand and placing the delivery device with the skin-contacting surface of the housing against the skin such that the least one targeting guide aligns with an intended injection site;
operating an unlocking system by rotating a part of the delivery device relative to the held portion of the housing, whereby an activation button disposed at the second end shifts axially away from the housing; and
without pressing the delivery device housing toward the injection site with any predetermined force by the one hand holding the housing portion, with another hand plunging the activation button toward the housing in the axial direction to trigger an advancing assembly within the device that first automatically advances the needled syringe from the first position to the second position, and that second automatically advances the syringe piston toward the second end.

2. The method of claim 1 wherein the step of operating the unlocking system is performed while the delivery device is positioned with the skin contacting surface of the housing against the skin.

3. The method of claim 1 wherein the at least one targeting guide comprises the first region of the skin-contacting surface of the housing.

4. The method of claim 1 wherein the step of holding the housing portion with one hand involves, after placing the delivery device with the skin-contacting surface of the housing against the skin such that the least one targeting guide aligns with an intended injection site, adjusting the holding hand to cover at least substantially all of the region of an axial length of the housing which is transparent to otherwise allow visibility of the needled syringe.
